(19) United States
(12) Patent Application Publication
(10) Pub. No.: US 2026/0265557 A1
(43) Pub. Date: Sep. 10, 2026

(54) SUBSTRATE WITH AN AROMA PRINTED THEREON

(71) Applicants: International Flavors & Fragrances Inc., New York, NY (US); The Additive Advantage LLC, Woburn, MA (US)

(72) Inventors: Manyan Wang, Woburn, MA (US); Adam Toth, Washington, NJ (US); Franklin Pringgosusanto, Manalapan, NJ (US)

(21) Appl. No.: 19/165,863

(22) PCT Filed: Mar. 26, 2024
(86) PCT No.: PCT/US2024/021465

§ 371 Date: Sep. 16, 2025

Related U.S. Application Data
(60) Provisional application No. 63/454,759, filed on Mar. 27, 2023.
(60) Provisional application No. 63/454,764, filed on Mar. 27, 2023.

(51) Int. Cl. C09D 11/38; A61L 9/012; B32B 7/06; B32B 7/12

(52) U.S. Cl. CPC C09D 11/38; A61L 9/012; B32B 7/06; B32B 7/12

(57) ABSTRACT

This disclosure relates to a composition for the delivery of an aroma. The composition includes a substrate and an aroma emulsion printed and dried on a surface of the substrate, wherein the aroma emulsion contains (i) an aroma, (ii) a carrier containing a modified starch and a disaccharide and/or a disaccharide alcohol, and optionally (iii) a retention aid. This disclosure also relates to a method of producing such composition. This disclosure also relates to a consumer product or consumer package containing such composition.

### FIELD OF THE DISCLOSURE

The present disclosure relates to a substrate with an aroma emulsion printed and dried on its surface. The present disclosure also relates to methods of producing such printed and dried substrate. The present disclosure also relates to consumer product or consumer package comprising such printed and dried substrate.

### BACKGROUND OF THE DISCLOSURE

Prolonged and controlled release of flavors and/or fragrances from a substrate is a trend in the aroma delivery industry. Conventionally, the substrate is treated with an aroma oil by spraying, coating, or dipping. Alternatively, filaments are treated with an aroma oil prior to fabrication. However, the imparted aroma effect is often short-lived.

Further, the combination of an aroma with certain active ingredients such as surfactants can result in poor aroma retention. While microencapsulation of aroma oils can provide protection of the aroma, microencapsulation can be costly and involves additional processes.

An effective, cost-efficient, and sustainable technology for controlled release of a flavor and/or fragrance from a substrate is therefore needed, which protects the flavor and/or fragrance, and releases the flavor and/or fragrance under predetermined conditions.

### BRIEF SUMMARY OF THE DISCLOSURE

The present disclosure provides a composition for the delivery of an aroma (e.g., flavor and/or fragrance). The composition comprises a substrate and an aroma emulsion printed and dried on a surface of the substrate, wherein the aroma emulsion comprises (i) an aroma, (ii) a carrier comprising a modified starch and a disaccharide and/or a disaccharide alcohol, and optionally (iii) a retention aid.

### BRIEF DESCRIPTION OF THE FIGURES

Embodiments are illustrated in the accompanying figures to improve understanding of concepts as presented herein.

FIG. 1 illustrates different conditions in which an aroma printed on a substrate may be released. The printed aroma may be used in closed environment applications to consistently supply aroma to a headspace by slow diffusion. Alternatively, aroma may be released by repeated activation via abrasion, sudden burst release by water addition, controlled release by heating, or activation by delaminating a protective layer. Also, aroma may be released via various combinations of these methods.

FIG. 2A and FIG. 2B illustrate patterns of printed emulsion dots on the surface of a substrate. Two different emulsions may be printed side-by-side on segments of the surface of a substrate covering large areas (FIG. 2A) or in alternating rows (FIG. 2B). The aromas in each dried emulsion may be released simultaneously in response to the same trigger (e.g., water) or be released individually by different triggers.

FIG. 3 shows the fragrance retention under ambient conditions of fragrance emulsion printed and dried on a substrate sheet as compared to neat fragrance oil pipetted on a substrate sheet. In FIG. 3, “FR” means fragrance, “Print” means printed and dried fragrance emulsion, and “Neat Oil” means neat fragrance oil.

FIG. 4 shows average fragrance retention in freshly prepared and 8-week-old printed samples having dot sizes of 10-70%. The percent dot size is a printer setting, which modulates the percentage of voltage applied to control the opening of the nozzle. 100% means the nozzle is fully open. The dot sizes have the order of increase: 10%<20%<40%<70%, 10% dot size corresponds to 0.256 mg wet dot weight, 20% dot size corresponds to 0.319 mg wet dot weight, 40% dot size corresponds to 0.627 mg wet dot weight, and 70% dot size corresponds to 1.061 mg wet dot weight. In FIG. 4, “Fresh” means freshly prepared printed and dried fragrance emulsion, and “8-week” means the printed and dried fragrance emulsion stored for 8 weeks.

FIG. 5A and FIG. 5B show the fragrance retention of individual fragrance ingredients (p1-p26) under ambient condition in printed fragrance emulsion (FIG. 5A) as compared to neat fragrance oil pipetted on the substrate (FIG. 5B).

FIG. 6A and FIG. 6B show sensory tests of printed fragrance emulsion samples compared to neat fragrance oil samples before (FIG. 6A) and after (FIG. 6B) the abrasion tests. In FIG. 6A and FIG. 6B, “Print sticker” means the sticker substrate with the fragrance emulsion printed and dried thereon, and “Neat oil sticker” means the sticker substrate with the neat fragrance oil pipetted thereon.

### DETAILED DESCRIPTION

The foregoing general description and the following detailed description are exemplary and explanatory only and are not restrictive of the invention, as defined in the appended claims. Other features and benefits of any one or more of the embodiments will be apparent from the following detailed description, and from the claims.

As used herein, the terms “comprise”, “comprising”, “include”, “including,” “have”, “having”, “contain”, “containing” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8”, “3 to 10”, “2 to 7”, “1.5 to 6”, “3.4 to 7.8”, “1 to 2 and 7-10”, “2 to 4 and 6 to 9”, “1 to 3.6 and 7.2 to 8.9”, “1-5 and 10”, “2 and 8 to 10”, “1.5-4 and 8”, and the like.

The present disclosure illustratively described herein suitably may be practiced in the absence of any element or elements, limitation or limitations that are not specifically disclosed herein. While compositions and methods are described herein in terms of “comprising” various components or steps, the compositions and methods also can “consist essentially of” or “consist of” the various components or steps, unless stated otherwise.

All parts, percentages and proportions referred to herein and in the claims are by weight unless otherwise indicated.

Before addressing details of embodiments described below, some terms are defined or clarified.

The term “consumer product”, as used herein, means a product which is typically used by a consumer, such as automotive products, bathroom products, kitchen products, laundry products, paper products, personal products, and sport products. Illustrative examples of such “consumer products” include laundry products (e.g., dryer sheets or detergent sheets, dye trapping sheets), odor reducing or eliminating materials (e.g., shoe inserts), paper towels, toilet tissue, paper plates, paper cups, writing paper, disposable dusting sheets, feminine hygiene products (e.g., tampons, pads, adult incontinence products, interlabile products, and the like), diapers, disposable wipes, aluminum foil, polymeric kitchen films, sponges, disposable plates, disposable cups, disposable tableware, scouring pads, water filters, water filter cartridges, tile cleaners, toilet cleaners, floor cleaners, automotive air fresheners, razors, makeup remover, garbage bags, food storage bags, and the like.

The term “consumer package” or “consumer packaging”, as used herein, means the receptacle (e.g., container, bag, box) containing the consumer product. Illustrative examples of such “consumer packaging” include drink cans and bottles, shoe boxes, package inserts or literature, food containers (e.g., chip bags, cereal boxes, cans, frozen meal containers) and the like.

The term “substrate”, as used herein, means a material with at least one surface capable of receiving an emulsion by printing. The substrate can be of any size and shape. The size and shape of the substrate may be dependent upon the product or package being produced or application of the printed substrate. In some aspects, the substrate is a flat material, convex material, or concave material. In other aspects, the substrate may be a woven material or non-woven material. In further aspects, the substrate may be swellable, non-swellable, porous, non-porous, charged, uncharged, hydrophobic, hydrophilic, fibrous, nonfibrous, dissolvable, non-dissolvable, erodible, or non-erodible. Moreover, depending on the material, e.g., porosity or charge, the printed emulsion may remain on the surface of substrate, may be embedded in the surface of the substrate, or may be absorbed by the substrate.

The term “emulsion”, as used herein, means a fluidic state which exists when a first fluid is dispersed in a second fluid that is typically immiscible with the first fluid, e.g., oil droplets dispersed in an aqueous solution.

The term “on a moisture-free basis”, as used herein with reference to a specific weight, means the weight of a material after it has been dried to completely remove all moisture, e.g., the moisture content of the material is 0%. Specifically, the weight on a moisture-free basis of a material can be obtained by weighing the material after the material has been placed in a 45° C. oven until the material reaches a constant weight.

The term “wt %”, as used herein, means percentage by weight.

As used herein, the terms “g,” “mg,” and “μg” refer to “gram,” “milligram,” and “microgram,” respectively. The terms “L” and “mL” refer to “liter” and “milliliter,” respectively. The terms “m”, “cm”, “mm”, “μm” and “nm” refer to “meter”, “centimeter”, “millimeter”, “micrometer” and “nanometer” respectively. The term “ft” refers to “foot” or “feet”, the term “min” refers to “minute” or “minutes”, and the term “kD” refers to “kilodalton” or “kilodaltons”.

The present disclosure provides a composition for delivering an aroma (e.g., flavor and/or fragrance) in a consumer product or consumer package. The composition comprises a substrate and an aroma emulsion printed and dried on a surface of the substrate, wherein the aroma emulsion comprises (i) an aroma, (ii) a carrier comprising (iia) a modified starch and (iib) a disaccharide and/or a disaccharide alcohol, and optionally (iii) a retention aid. Advantageously, the present disclosure provides a sustainable and cost-efficient technology for controlled delivery of aroma from consumer products or packaging, with high retention of aroma during processing, storage, and ambient environmental exposure. It has been surprisingly discovered that the combination of modified starch with disaccharide and/or disaccharide alcohol can significantly increase the aroma retention in the composition. It has also been surprisingly discovered that the substrate surface made of polymer selected from the group of polyester, polyurethane, polypropylene, polyethylene, cellulose, and combinations thereof can further increase the aroma retention in the composition. Thus, the technology in the present disclosure may eliminate expensive packaging otherwise needed to protect an aroma from loss during storage and transportation.

The aroma emulsion comprises (i) an aroma, (ii) a carrier comprising (iia) a modified starch and (iib) a disaccharide and/or a disaccharide alcohol, and optionally (iii) a retention aid. In some embodiments, the aroma comprises or is a flavor and/or a fragrance. In some embodiments, the aroma comprises or is a flavor. In some embodiments, the aroma comprises or is a fragrance. In some embodiments, the fragrance includes fine fragrances, odor masking fragrances, and medicinal aromatics. In some embodiments, the aroma is selected from the group of flavors, fragrances, malodor counteractive agents, pro-fragrances, and combinations thereof. The aroma may be used alone or in combination with other active ingredients, for example, deodorants, moisture-absorbents, antioxidants, antimicrobials, antivirals, pest deterrents, taste modulators, and/or artificial sweeteners.

A variety of flavors and fragrances may be used in the present disclosure. Flavors and fragrances may be chosen from synthetic flavors and fragrances, oils and oil extracts derived from plants, leaves, flowers, fruits, and combinations thereof. Representative flavor oils include, but are not limited to, spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit, and fruit essences including apple, pear, peach, strawberry, watermelon, raspberry, cherry, plum, pineapple, apricot and so forth. Representative fragrances include, but are not limited to, animal fragrances such as musk oil, civet, castoreum, ambergris, plant fragrances such as nutmeg extract, cardamom extract, ginger extract, cinnamon extract, patchouli oil, geranium oil, orange oil, mandarin oil, orange flower extract, cedarwood, vetiver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, carrot seed extract, jasmine extract, olibanum extract, rose extract, and mixtures thereof. These flavors and fragrances can be used individually or in combination.

In some embodiments, after drying of the aroma emulsion on the surface of the substrate, the printed and dried aroma emulsion comprises at least 5 wt %, 10 wt %, 15 wt %, 20 wt %, 25 wt %, 30 wt %, 35 wt %, 40 wt %, 45 wt %, 50 wt %, 55 wt %, or 60 wt % aroma, based on the weight of the printed and dried aroma emulsion on a moisture-free basis. In some embodiments, the printed and dried aroma emulsion comprises no more than 75 wt %, 70 wt %, 65 wt %, 60 wt %, 55 wt %, 50 wt %, 45 wt %, or 40 wt % aroma, based on the weight of the printed and dried aroma emulsion on a moisture-free basis. In some embodiments, the amount of the aroma in the printed and dried aroma emulsion is in a range of from 5 wt % to 70 wt %, from 10 wt % to 60 wt %, from 20 wt % to 50 wt %, from 25 wt % to 50 wt %, from 25 wt % to 60 wt %, from 40 wt % to 70 wt %, from 45 wt % to 70 wt %, from 50 wt % to 70 wt %, or from 55 wt % to 70 wt %, based on the weight of the printed and dried aroma emulsion on a moisture-free basis.

The aroma can be encapsulated (i.e., encapsulated aroma) or unencapsulated (i.e., neat aroma). As used herein, the term “encapsulated aroma” means an aroma which is encapsulated in a microcapsule, and the term “neat aroma” means an aroma which is unencapsulated. In some embodiments, the aroma is a combination of neat aroma and encapsulated aroma, and the weight ratio of the neat aroma to the encapsulated aroma is from 10:1 to 1:1, from 8:1 to 2:1, from 7:1 to 2:1, from 6:1 to 3:1, or from 5:1 to 3:1. In some embodiments, the aroma comprises or is neat aroma.

In some embodiments, the aroma emulsion is an oil-in-water emulsion, and the neat aroma is present in the emulsion as oil droplets. In some embodiments, the aroma oil droplets present in the aroma emulsion have a mean oil droplet size of less than about 5 microns, less than about 4 microns, less than about 3 microns, less than about 2 microns, less than about 1 micron, less than about 0.9 micron, less than about 0.8 micron, less than about 0.7 micron, or less than about 0.6 micron, as determined by conventional methods such as laser diffraction, electrical pulse counting or ultrasonic spectrometry. It has been surprisingly discovered that having mean aroma oil droplet size below 1 micron can significantly increase the aroma retention in the composition.

The carrier in the aroma emulsion comprises a combination of (a) a modified starch and (b) a disaccharide and/or a disaccharide alcohol. In some embodiments, the carrier comprises a modified starch and a disaccharide. The modified starch in the present disclosure is a chemically modified starch. Such modifications include, but are not limited to acid treatment, alkaline treatment, bleaching, oxidation, enzyme treatment, acetylation, phosphorylation, or a combination thereof. In some embodiments, the modified starch is selected from the group of cationic starches, hydroxyethyl starches, carboxymethylated starches, and combinations thereof. In some embodiments, the modified starch comprises or is an octenyl succinic anhydride (OSA) modified starch, such as those sold under the tradenames CAPSUL® and HI-CAP® 100. In some embodiments, the modified starch comprises or is starch sodium octenyl succinate (E1450). In some embodiments, the modified starch has weight average molecular weight (M_{w}) of at least 5 kD (kilodaltons), at least 10 kD, at least 15 kD, at least 20 kD, at least 30 kD, or at least 50 kD. In some embodiments, the modified starch has M_{w} of no more than 800 kD, no more than 700 kD, no more than 600 kD, no more than 500 kD, no more than 400 kD, no more than 300 kD, or no more than 200 kD.

In some embodiments, the disaccharide is selected from the group of maltose, sucrose, fructose, trehalose, lactose, and combinations thereof. In some embodiments, the disaccharide is selected from the group of maltose, sucrose, trehalose, and combinations thereof. In some embodiments, the disaccharide comprises or is maltose. A disaccharide alcohol is a derivative of a disaccharide that has been hydrogenated. In some embodiments, the disaccharide alcohol is selected from the group of isomalt, mannitol, maltitol, xylitol, lactitol, and combinations thereof. In some embodiments, the disaccharide alcohol comprises or is isomalt and/or maltitol. In some embodiments, the disaccharide alcohol comprises or is isomalt.

In some embodiments, the carrier comprises at least 3 wt %, 4 wt %, 5 wt %, 8 wt %, 10 wt %, 15 wt %, 20 wt %, 25 wt %, 30 wt %, 35 wt %, 40 wt %, 45 wt %, 50 wt %, or 55 wt % disaccharide and/or disaccharide alcohol, based on the weight of the carrier. In some embodiments, the carrier comprises no more than 90 wt %, 85 wt %, 80 wt %, 75 wt %, 70 wt %, 65 wt %, 60 wt %, 55 wt %, 50 wt %, or 45 wt % disaccharide and/or disaccharide alcohol, based on the weight of the carrier. In some embodiments, the carrier comprises from 5 wt % to 90 wt %, from 5 wt % to 70 wt %, from 5 wt % to 60 wt %, from 5 wt % to 50 wt %, from 10 wt % to 70 wt %, from 10 wt % to 60 wt %, or from 10 wt % to 50 wt % disaccharide and/or disaccharide alcohol, based on the weight of the carrier. In some embodiments, the disaccharide and/or disaccharide alcohol is maltose.

In some embodiments, the carrier comprises at least 10 wt %, 15 wt %, 20 wt %, 25 wt %, 30 wt %, 35 wt %, 40 wt %, 45 wt %, 50 wt %, or 55 wt % modified starch, based on the weight of the carrier. In some embodiments, the carrier comprises no more than 97 wt %, 96 wt %, 95 wt %, 92 wt %, 90 wt %, 85 wt %, 80 wt %, 75 wt %, 70 wt %, 65 wt %, 60 wt %, 55 wt %, 50 wt %, or 45 wt % modified starch, based on the weight of the carrier. In some embodiments, the carrier comprises from 10 wt % to 95 wt %, from 30 wt % to 95 wt %, from 40 wt % to 95 wt %, from 50 wt % to 95 wt %, from 30 wt % to 90 wt %, from 40 wt % to 90 wt %, or from 50 wt % to 90 wt % modified starch, based on the weight of the carrier. In some embodiments, the modified starch is OSA modified starch.

In some embodiments, the carrier in the aroma emulsion can further comprise a starch (e.g., rice starch, potato starch, corn starch), a dextrin, a maltodextrin, a gum (e.g., arabic gum, xanthan gum, guar gum, locust bean gum, carrageenan), a cellulose, a cellulose derivative (e.g., hydroxypropyl methylcellulose (HPMC), methylcellulose (MC), hydroxypropyl cellulose (HPC), hydroxyethyl cellulose (HEC) and carboxymethyl cellulose (CMC)), a pectin, a chitin, a chitosan, or a combination thereof. In some embodiments, the carrier can further comprise dextran, pullulan, fructan, mannan, inulin, polydextrose, fleximer (a ring-opened form of dextran), or a combination thereof.

In some embodiments, the weight ratio of the carrier to the aroma is at least 1:4, 1:3, 1:2.5, 1:2.3, 1:2, 1:1.5, or 1:1. In some embodiments, the weight ratio of the carrier to the aroma is no more than 10:1, 5:1, 4:1, 3:1, 2:1, or 1.5:1. In some embodiments, the weight ratio of the carrier to the aroma is in a range of from about 1:4 to about 10:1, from about 1:2.4 to about 4:1, or from about 1:2.3 to about 1:1.5.

In some embodiments, the aroma emulsion comprises the retention aid. As used herein, the term “retention aid” means a material that creates or increases an affiliation between the aroma and/or carrier of the emulsion and the substrate. In this respect, the retention aid may improve the performance of the aroma, e.g., by decreasing loss of the aroma from the composition over time or during storage. In some embodiments, the retention aid is selected from the group of sugars, plasticizers, and combinations thereof. In some embodiments, the retention aid is a combination of a sugar and a plasticizer. In some embodiments, the sugar in the retention aid is different from the disaccharide in the carrier. Exemplary sugars of use as retention aids include, but are not limited to, lactose, levulose, glucose, sucrose, fructose, maltose, trehalose, cellobiose, chitobiose, ribose, arabinose, pentose, xylose, galactose, dextrose, and isomaltose. Examples of suitable plasticizers include, but are not limited to, esters such as phthalate esters (diethyl, dibutyl), ortho-phthalates, terephthalates, sebacates, citrate esters (triethyl, acetyl triethyl, acetyl tributyl), triacetin, adipates, azelates, benzoates, trimellitates, polyols such as glycerol (glycerin), propylene glycol, polyethylene glycols (PEG), and glycerides such as acetylated monoglycerides.

In some embodiments, the printed and dried aroma emulsion comprises no more than 10 wt %, 8 wt %, 6 wt %, 4 wt %, 2 wt %, 1 wt %, 0.5 wt %, or 0.1 wt % retention aid, based on the weight of the printed and dried aroma emulsion on a moisture-free basis. In some embodiments, the printed and dried aroma emulsion comprises at least 0.05 wt %, 0.5 wt %, 1 wt %, 3 wt %, or 5 wt % retention aid, based on the weight of the printed and dried aroma emulsion on a moisture-free basis. In some embodiments, the aroma emulsion is free of the retention aid.

In some embodiments, the printed and dried aroma emulsion comprises, by weight based on the weight of the aroma emulsion on a moisture-free basis, from about 5 wt % to about 70 wt % aroma, at least about 20 wt % carrier, and less than about 10 wt % retention aid. In some embodiments, the printed and dried aroma emulsion comprises, by weight based on the weight of the aroma emulsion on a moisture-free basis, from about 20 wt % to about 60 wt % (preferably from 25 wt % to 60 wt % or from 30 wt % to 50 wt %) aroma, from about 20 wt % to about 60 wt % (preferably from 30 wt % to 50 wt %) carrier, and from 0 wt % to 10 wt % (preferably from 0.5 wt % to 5 wt %, from 1 wt % to 5 wt %, from 0.5 wt % to 3 wt %, or from 3 wt % to 5 wt %) retention aid.

In addition to the aroma, carrier and optional retention aid, the aroma emulsion may further comprise one or more additional additives such as: additional actives (e.g., antioxidants, anti-inflammatory agents, anesthetics, analgesics, anti-fungal agents, antibiotics, anti-viral agents, anti-parasitic agents, enzymes, co-enzymes, anti-histamines, and/or chemotherapeutic agents), stabilizers, emulsifiers (e.g., sodium lauryl sulfate, lecithins, sucrose esters, polysorbates, quillaja extract, and/or saponins), binders, sweeteners (e.g., Stevia rebaudiana extracts, rebaudioside A (Reb A), stevioside, Reb D, erythritol, xylitol, mannitol, sorbitol, inositol, aspartame, sucralose, and/or neotame), solid particles (e.g., silica and/or MCC), acidulants (e.g., citric acid, malic acid, succinic acid, acetic acid, hydrochloric acid, adipic acid, tartaric acid, fumaric acid, phosphoric acid, lactic acid, sodium acid pyrophosphate, and salts thereof), vitamins (e.g., vitamin A and its analogs and derivatives, vitamin E, vitamin C, vitamin B3, alpha hydroxy acids, and/or beta hydroxy acids), dyes, colorants or pigments (lactoflavin, a carotene, curcumin, lycopene, a xanthophyll, cochineal red A, beetroot red, and/or phthalocyanine green), microcapsules, waxes, and a combination thereof. In some aspects, the amount of the additional additive is from 0.1% to 50% (e.g., from 0.2 to 40%, from 0.3 to 30%, from 0.4 to 20%, or from 0.5 to 10%) by weight based on the weight of the aroma emulsion on a moisture-free basis.

In some embodiments, the aroma emulsion further comprises water. In some embodiments, the aroma emulsion comprises no more than 90 wt %, 80 wt %, 70 wt %, 65 wt %, 60 wt %, 55 wt %, 50 wt %, 45 wt %, 40 wt %, or 35 wt % water, based on the weight of the aroma emulsion. In some embodiments, the aroma emulsion comprises at least 20 wt %, 25 wt %, 30 wt %, 35 wt %, 40 wt %, 45 wt %, 50 wt %, 55 wt %, 60 wt %, or 65 wt % water, based on the weight of the aroma emulsion. In some embodiments, the aroma emulsion comprises from about 20 wt % to about 80 wt %, from about 32 wt % to about 80 wt %, from about 40 wt % to about 70 wt %, or from about 50 wt % to about 65 wt % water, based on the weight of the aroma emulsion.

In some embodiments, the aroma emulsion (before drying) has a viscosity of at least 10 cP (centipoise), 20 cP, 40 cP, 50 cP, 100 cP, 200 cP, 300 cP, 400 cP, 500 cP, 600 cP, 700 cP, 800 cP, 900 cP, or 1000 cP at room temperature (about 20° C.). In some embodiments, the aroma emulsion (before drying) has a viscosity of no more than 10000 cP, 9000 cP, 8000 cP, 7000 cP, 6000 cP, 5000 cP, 2000 cP, 1000 cP, 500 cP, or 100 cP at room temperature (about 20° C.). In some embodiments, the aroma emulsion (before drying) has a viscosity in a range of from 5 cP to 100 cP at room temperature (about 20° C.).

In some embodiments, the printed and dried aroma emulsion comprises no more than 15%, 12%, 10%, 8%, 6%, 5%, or 4% water, by weight based on the weight of the aroma emulsion. In some embodiments, the printed and dried aroma emulsion comprises from 4% to 10%, or from 4% to 8% water, by weight based on the weight of the aroma emulsion. In some embodiments, the printed and dried aroma emulsion has a water activity (at 25° C.) of from 0.1 to 0.6, from 0.2 to 0.5, or from 0.2 to 0.4. The term “water activity”, as used herein with respect to a substance, means the partial vapor pressure of water in the substance at a given temperature divided by the partial vapor pressure of pure water at the same temperature.

The surface of the substrate can be made of a polymer selected from the group of polyester, polyurethane, polysaccharide (e.g., pullulan, cellulose, regenerated cellulose such as rayon), polypropylene, polyethylene, polyvinyl alcohol (PVOH), gelatin, alginate, starch (e.g., a modified starch or natural starch such as tapioca starch), hydroxypropyl methylcellulose (HPMC), silk (i.e., polypeptides such as sericin and/or fibroin), nylon (i.e., aliphatic or semi-aromatic polyamides), wool (i.e., keratin polymer), and combinations thereof. In some embodiments, the surface of the substrate is made of leather, glass, metal, ceramic, rubber, paper, cotton, rayon, plastic film, food product (e.g., chewing gum, snack or cracker), or a combination thereof. It has been surprisingly discovered that the composition comprising an aroma emulsion printed and dried on the substrate surface made of a polymer selected from the group of polyester, polyurethane, polypropylene, polyethylene, cellulose, and combinations thereof can have improved aroma retention in the composition, comparing with a corresponding composition having a substrate surface made of certain other polymers (e.g., PVOH). Accordingly, preferably the surface of the substrate is made of a polymer selected from the group of polyester, polyurethane, polypropylene, polyethylene, cellulose, and combinations thereof.

In some embodiments, the printed and dried aroma emulsion comprises no more than 50 wt %, 45 wt %, 40 wt %, 35 wt %, 30 wt %, 25 wt %, or 20 wt % aroma, based on the weight of the printed and dried aroma emulsion on a moisture-free basis; the carrier comprises from 5 wt % to 80 wt %, from 5 wt % to 70 wt %, from 10 wt % to 70 wt %, from 10 wt % to 60 wt %, from 10 wt % to 50 wt %, or from 10 wt % to 40 wt % maltose, based on the weight of the carrier; and the surface of the substrate is made of a polymer selected from the group of PVOH, HPMC, gelatin, pullulan, methylcellulose, polyvinylpyrrolidone (PVP), pectin, and combinations thereof. In some embodiments, the surface of the substrate is made of a polymer selected from the group of PVOH, HPMC, and combinations thereof. It has been discovered that such embodiments can achieve high aroma retention.

In some embodiments, the printed and dried aroma emulsion comprises from 10 wt % to 50 wt %, from 20 wt % to 50 wt %, from 20 wt % to 40 wt %, from 25 wt % to 50 wt %, from 15 wt % to 45 wt %, from 25 wt % to 45 wt %, from 15 wt % to 40 wt %, from 25 wt % to 40 wt %, or from 15 wt % to 35 wt % aroma, based on the weight of the printed and dried aroma emulsion on a moisture-free basis; the carrier comprises from 5 wt % to 90 wt %, from 5 wt % to 80 wt %, from 5 wt % to 70 wt %, from 5 wt % to 60 wt %, from 5 wt % to 50 wt %, from 10 wt % to 80 wt %, from 10 wt % to 70 wt %, from 10 wt % to 60 wt %, from 10 wt % to 50 wt %, or from 10 wt % to 40 wt % maltose, based on the weight of the carrier; and the surface of the substrate is made of a polymer selected from the group of polyester, polyurethane, polypropylene, polyethylene, cellulose, and combinations thereof. It has been discovered that such embodiments can achieve high aroma retention.

In some embodiments, the printed and dried aroma emulsion comprises from 40 wt % to 70 wt %, from 45 wt % to 70 wt %, from 50 wt % to 70 wt %, or from 55 wt % to 70 wt % aroma, based on the weight of the printed and dried aroma emulsion on a moisture-free basis; the carrier comprises from 5 wt % to 80 wt %, from 5 wt % to 70 wt %, from 5 wt % to 60 wt %, from 10 wt % to 80 wt %, from 10 wt % to 70 wt %, from 10 wt % to 60 wt %, from 10 wt % to 50 wt %, or from 10 wt % to 40 wt % maltose, based on the weight of the carrier; and the surface of the substrate is made of a polymer selected from the group of polyester, polyurethane, polypropylene, polyethylene, cellulose, and combinations thereof. It has been discovered that such embodiments can achieve high aroma retention.

In some embodiments, the printed and dried aroma emulsion comprises from 60 wt % to 70 wt % aroma, based on the weight of the printed and dried aroma emulsion on a moisture-free basis; the carrier comprises from 10 wt % to 65 wt %, from 20 wt % to 65 wt %, from 30 wt % to 65 wt %, from 40 wt % to 65 wt %, from 45 wt % to 65 wt %, or from 50 wt % to 65 wt % maltose, based on the weight of the carrier; and the surface of the substrate is made of a polymer selected from the group of polyester, polyurethane, polypropylene, polyethylene, cellulose, and combinations thereof. It has been discovered that such embodiments can achieve high aroma retention.

The substrate can be single layered or multilayered. In some embodiments, the substrate is multilayered, that is, the substrate has two or more layers. Each of the two or more layers may be composed of the same or a different material (e.g., polymer). Whether single or multilayered, each layer of the substrate may be composed of one or more materials and may be in the form of a film, tape, sheet, wipe, bag, sticker, food package, insert, or pad.

In some embodiments, the aroma emulsion printed and dried on the surface of the substrate may be protected (to prevent aroma release or loss) by applying an adhesive removable cover film/sheet, or an adhesive removable backing layer to the printed surface of the substrate. For example, a fine fragrance may be printed on a sticker and a cover sheet pretreated with a pressure sensitive adhesive can be applied to the surface of the sticker comprising the printed fragrance so that removal of the cover sheet allows a customer to sample a perfume. Accordingly, in some embodiments, the composition further comprises an adhesive cover film or backing layer on the printed surface of the substrate.

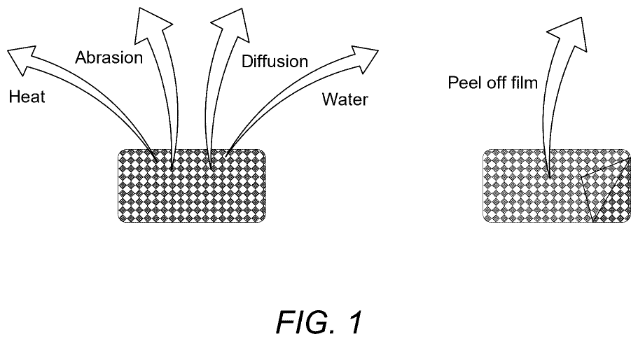

The release of aroma can be controlled by a variety of application conditions, e.g., heat, diffusion, repeated abrasion, water and/or humidity, removing an adhesive removable cover film from the printed surface, or by various combinations thereof. (FIG. 1) The release of aroma can also be controlled by adjusting printing parameters such as dot size, dot spreading, and/or the composition of the aroma emulsion.

The present disclosure also provides a method of producing the composition described in the present disclosure to create a desired consumer experience. The method comprises: (a) providing the aroma emulsion comprising (i) the aroma, (ii) the carrier comprising the modified starch and the disaccharide and/or the disaccharide alcohol, and optionally (iii) the retention aid; (b) printing the aroma emulsion on the surface of the substrate; and (c) drying the printed aroma emulsion on the surface of the substrate. In some embodiments, the aroma emulsion is printed on the surface of the substrate in a predetermined pattern or design, that is, the location and amount of the aroma emulsion to be printed on the surface of the substrate is determined prior to printing. The aroma emulsion can be printed as letters, numbers, or geometric shapes including but not limited to, squares, circles or dots, triangles, rectangles, hexagons, etc. A design may be characters, words, or text as well as other features such as graphics, images, or logos. In some aspects, the aroma emulsion is printed as individual dots that collectively provide a shape or design.

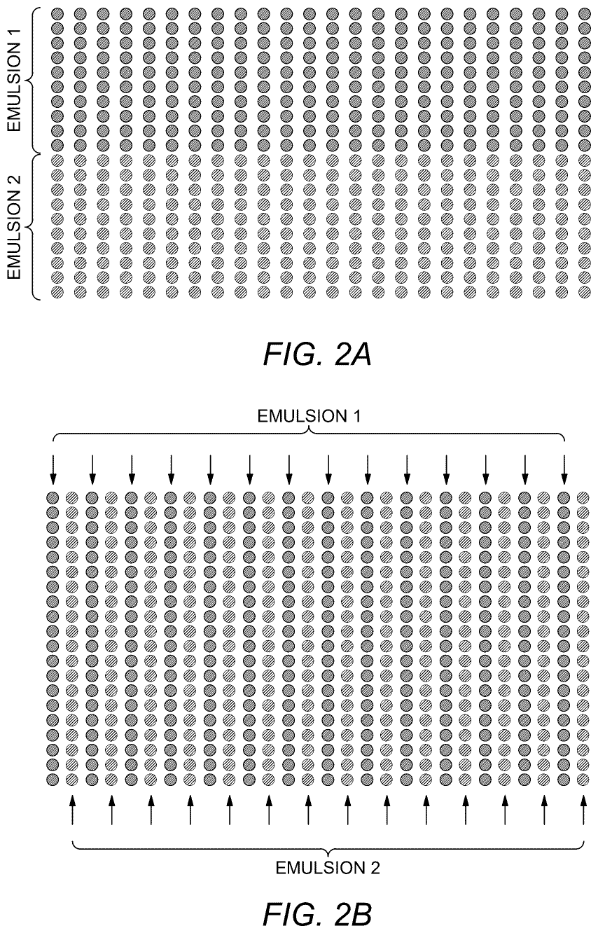

In some embodiments, the aroma emulsion is printed on the surface of the substrate in an array (e.g., an array of dots), wherein the array comprises a dot pattern evenly distributed and spaced center point-to-center point of from 1 mm to 30 mm between dots. (See FIG. 2). In some embodiments, the dots have center point-to-center point spacing of from 1 mm to 20 mm, or from 1 mm to 10 mm between dots. In some embodiments, the dots have substantially the same diameter and center point-to-center point spacing of from 1.1 times the diameter to 3 times the diameter, or from 1.1 times the diameter to 2 times the diameter, or from 1.1 times the diameter to 1.5 times the diameter.

In some embodiments, the aroma emulsion can be prepared or provided via the following steps, that is, the step (a) can comprise the following steps: (a1) preparing an aqueous phase by dissolving and/or dispersing the carrier and optionally the retention aid with water, (a2) preparing an oil phase comprising the aroma, and (a3) mixing the oil phase with the aqueous phase to generate an oil-in-water emulsion. In some embodiments, the oil phase and the aqueous phase is first mixed to form a coarse emulsion, and the coarse emulsion is then subject to shear forces (e.g., a high-speed homogenizer) to generate the aroma emulsion that is to be printed. In some embodiments, the aroma emulsion is generated at a shear rate in the range of from 10000 rpm to 30000 rpm, or from 12000 rpm to 25000 rpm, for a period of from about 3 minutes to 15 minutes, or from about 5 minutes to 10 minutes. In some embodiments, the generated aroma emulsion is diluted (e.g., with water or aqueous solution or suspension) and/or added with an additional additive (e.g., solid particle, surfactant, dye, colorant or pigment, gum, and/or additional active, etc.) prior to being printed on the surface of the substrate.

In step (b), the aroma emulsion is printed on the surface of the substrate. In some embodiments, the aroma emulsion can be printed, dropped, or deposited directly onto the surface of the substrate in a controlled and precise manner in a predefined pattern, e.g., a dot pattern or design. In this respect, “printing” in accordance with the present method is distinct from spraying (emulsion on substrate), coating (emulsion on substrate), atomizing (emulsion on substrate), or dipping (substrate in emulsion), which applies the emulsion to the substrate in an uncontrolled, nonuniform or random pattern (spraying or atomizing) or without uncoated areas on the surface of the substrate (coating or dipping). Moreover, in contrast to spraying, coating, atomizing or dipping, droplets of the aroma emulsion in the present method may be printed at a predetermined size to modulate one or more of the amount of aroma on the substrate, the amount of aroma released, dot spreading, or compatibility with the substrate.

A print assembly can be used to print the aroma emulsion on the surface of the substrate. The print assembly may be a non-contact print assembly, e.g., an inkjet-type print head or array of nozzles that drop or deposit the emulsion on a surface; or a contact print assembly, e.g., flat bed screen, rotary screen, reverse gravure, or flexography print assembly. In some embodiments, the print assembly is a non-contact print assembly. In some embodiments, the aroma emulsion is fed to a non-contact print assembly which prints or deposits the aroma emulsion in individual droplets on the surface of the substrate that directly receives the printed aroma emulsion. As used herein, the term “non-contact print assembly” means a print assembly which does not touch or is not in contact with the surface of the substrate during the printing process.

There are two types of jetting modes for non-contact print assemblies, continuous mode (CM) and drop-on-demand (DOD). In continuous mode jetting, an unbroken jet of fluid projects from an orifice; Rayleigh instabilities cause the jet to break up into individual droplets after a certain flight distance. In drop-on-demand mode, individual droplets are ejected from an orifice, usually as a result of a pulsed energy source.

In some embodiments, the aroma emulsion is printed or deposited in individual droplets on the surface of the substrate that directly receives the printed aroma emulsion. In some embodiments, the droplets have a mean droplet diameter of from 100 microns to 5 mm, or from 425 microns to 5 mm, or from 1 mm to 4 mm. In some embodiments, the droplets have substantially the same diameter. A droplet smaller than 425 microns (i.e., a particle passing through a 40 mesh sieve) is considered dust. In some embodiments, each individual droplet has a diameter of greater than 425 micron.

In step (c), the printed aroma emulsion is dried on the surface of the substrate. The printed aroma emulsion can be dried by, e.g., infrared, conduction, convection, or combination thereof. In some embodiments, the printed aroma emulsion is dried with air. In some embodiments, the printed aroma emulsion is dried with air at a temperature of from 30° C. to 160° C., or from 30° C. to 110° C., or from 40° C. to 100° C., or from 40° C. to 90° C. In some embodiments, the printed aroma emulsion is dried with air at a temperature of no more than 100° C. In some embodiments, the relative humidity (RH) of the air supplied to dry the printed aroma emulsion is no more than 35% RH, 15% RH, 7% RH, or 1% RH. In some embodiments, the printed aroma emulsion is dried with desiccated air. In some embodiments, the drying process can be conducted under an inert gas atmosphere. Examples of inert gases include nitrogen, carbon dioxide and noble gases such as argon. In some embodiments, the drying process is conducted under a nitrogen gas atmosphere. In practice, the inert gas atmosphere may still contain minor amounts of oxygen. Typically, the inert gas atmosphere has an oxygen level of less than 90 g oxygen per cubic meter of the atmosphere, or less than 50 g oxygen per cubic meter of the atmosphere, or less than 30 g oxygen per cubic meter of the atmosphere, or less than 25 g oxygen per cubic meter of the atmosphere, or less than 23 g oxygen per cubic meter of the atmosphere.

In some embodiments, the printed aroma emulsion is dried with a radiant heat source. Examples of suitable radiant heat sources include, but are not limited to, infrared and other light devices, electric radiant heaters, and radiant gas heaters. In some embodiments, the printed aroma emulsion is dried with an infrared light. In some embodiments, the printed aroma emulsion is dried with an infrared light in combination with conduction and/or convection.

In some embodiments, one or more of the method steps may be repeated. For example, the aroma emulsion (i.e., first emulsion) may be printed and dried on a surface of the substrate and a second emulsion may subsequently be printed on the same surface of the substrate and dried. In some embodiments, the aroma emulsion (i.e., first emulsion) is printed on a surface of the substrate, a second emulsion is printed on the same surface of the substrate, and both the aroma emulsion and the second emulsion are simultaneously dried. In some embodiments, the second emulsion is printed onto the aroma emulsion (i.e., first emulsion), that is, droplets of the second emulsion are printed onto the surface of the droplets of the aroma emulsion and the emulsions are dried. In some embodiments, the second emulsion is printed on the surface of the substrate at a location or area adjacent to where the aroma emulsion (i.e., first emulsion) is printed on the surface of the substrate. See FIG. 2A and FIG. 2B. Accordingly, the composition of the present disclosure can further comprise a second emulsion printed and dried on the surface of the substrate, wherein the second emulsion is printed onto or adjacent to the aroma emulsion.

Like the aroma emulsion (i.e., first emulsion), the second emulsion can also comprise (i) a second aroma, (ii) a second carrier comprising a second modified starch and a second disaccharide and/or a second disaccharide alcohol, and optionally (iii) a second retention aid. In some embodiments, at least one component of the aroma emulsion (i.e., first emulsion) and second emulsion is different. In such embodiments, the at least one component that is different can be the aroma, carrier or retention aid, or alternatively an additional additive. For example, the aroma emulsion and second emulsion may have the same aroma, carrier and retention aid, wherein the aroma emulsion comprises a blue pigment and the second emulsion comprises a red pigment. As another example, the aroma emulsion may comprise a carrier that has a different aroma release profile or trigger from the carrier of the second emulsion so that the aroma in the aroma emulsion and the aroma in the second emulsion are released at different times, different rates or by different triggers (e.g., water, heat, pH, etc.). In some embodiments, the second emulsion may comprise an ingredient that is incompatible with an ingredient in the aroma emulsion (i.e., first emulsion). As such, incompatible ingredients may be provided on the same substrate, but not in the same emulsion. The incompatibility of ingredients may include, but is not limited to, the potential for a reaction between the ingredients, an ingredient that may have a negative effect on aroma retention, and the like. By way of illustration, the abundance of surfactant used in cleaning items can affect the aroma retention such that printing the aroma and surfactant separately would be beneficial. Another example is the reaction of two ingredients when mixed: sodium nitrite reacts with citric acid under moisture to release nitric oxide, which may be used for meat storage. Thus, the composition of the present disclosure can accommodate two or more incompatible ingredients.

In some embodiments, the method of the present disclosure further comprises applying a retention aid onto the surface of the substrate. In such embodiments, the retention aid in the aroma emulsion and the retention aid applied to the substrate surface can be the same or different. In some embodiments, a retention aid is only applied onto the surface of the substrate but not added in the aroma emulsion.

In some embodiments, the method of the present disclosure further comprises a step of applying an adhesive removable cover film/sheet or adhesive removable backing layer to the printed surface of the substrate.

Printing the aroma emulsion in accordance with the present method provides a significant advantage over applying a neat aroma oil to a substrate. With neat aroma oils, volatile compounds vaporize quickly resulting in a rapid aroma release profile and a reduction in intensity over time. In comparison, the release of an aroma printed on a surface of a substrate in accordance with the present method may be modulated by the selection of the carrier, retention aid, dot size, and/or dot spreading to release the aroma under one or more specified conditions. As used herein, “dot spreading” refers to the area over which a printed dot spreads on the surface of the substrate. Alternatively stated, “dot spreading” refers to the contact area between the printed dot and the surface of the substrate.

The present disclosure also provides a consumer product or consumer package comprising the composition of the present disclosure. Examples of the consumer product or consumer package include, but are not limited to, a storage container or bag to impart a scent to the item being stored; a carry along item such as a sheet that releases a calming or invigorating fragrance on demand (e.g., by abrading the surface); a pillow insert with a calming fragrance (e.g., lavender) printed thereon to aid in sleep; a food packing insert that releases flavor to a food upon diffusion, heat and/or contact with moisture; a tea bag that releases flavor to a food upon heat and/or contact with moisture; personal care or hygiene products such as bath tissues or toilet paper that release a scent when applied to skin; a shoe insert that releases an odor masking and/or fragrance compound when inserted into the shoe or when compressed while walking; a trash bag that releases an odor masking and/or fragrance compound; fabric care sheets such as laundry sheets that release fragrance when exposed to the heat of a dryer; cleaning sheets, wipes or pads that release a scent when wiped (abraded) over a surface; cleaning pads that release scent when wetted; and abrasion activated car mats or entrance door mats that release fragrance when walked or stepped on.

Thus, the composition of the present disclosure may be used in a number of different scent applications in home care, personal care, beauty care, food deodorizing, fine fragrance, sleep, pest control, cleaning, scented wipes, and the like. In addition, the composition of the present disclosure may be used in food packaging, flavor delivery and other functional aroma delivery such as antimicrobial, antifungal or antioxidant. Still other applications may include medicinal aromatics to promote health and well-being, and environmental cleanliness.

Many aspects and embodiments have been described above and are merely exemplary and not limiting. After reading this specification, skilled artisans appreciate that other aspects and embodiments are possible without departing from the scope of the invention.

### EXAMPLES

The following non-limiting examples are provided to further illustrate the present invention and are not to be construed as limitations of the invention, as many variations of the present invention are possible without departing from its spirit or scope.

### Example 1: Test Methods

Activation Tests. The printed substrates (i.e., the substrate with the aroma emulsion printed and dried thereon) were tested for activated release of aroma by abrasion. Printed substrates were abraded repeatedly over a period of days and release of aroma and aroma profile were assessed.

Other forms of activation may also be tested including contact with water and lamination. Laminating a cover film over the printed surface of the substrate can completely hide the aroma and provide a burst of aroma when the cover film is peeled off. The cover film can be reapplied to the printed surface of the substrate and be repeatedly removed and reapplied for bursts of aroma release. In another aspect, water may be added to the printed substrate to dissolve the aroma emulsion and provide a burst of aroma. Further, exposing the aroma emulsion to high humidity may be used to release aroma in a continuous fashion.

Abrasion Test for Adhesion of Printed Aroma Emulsion on Substrate. An abrasion tester was used to quantitively test adhesion of printed aroma emulsion on substrates. The abrasion tester controlled the number of strokes and abrasion frequency. The printed substrate samples were taped to the support surface under abradant. The abradant sat inside its holder and was a piece of 0.25-inch diameter rubber disk having a hardness of 70 A. A certain weight was added on top to increase the abrasion strength. In use, the abradant was applied continuously in a back and forth motion across the sample for a pre-set number of strokes. The number of strokes the printed aroma emulsion survived with a certain loading weight provided an indication as to how well the printed aroma emulsion adhered to the substrate.

Aroma Retention Under Ambient Conditions. Aroma retention and olfactory sensory of printed aroma emulsions were compared with that of neat aroma oil. The neat aroma oil samples were prepared by pipetting 20 mg neat aroma oil onto nonwoven polyurethane substate in a pattern of dots similar to that of the printed aroma emulsion. The printed aroma emulsion also contained 20 mg of same aroma oil on the same size and kind of substrate. The samples were aged in a non-traffic room for up to 4 weeks under an ambient condition (room temperature, relative humidity 20-70%) and subsequently subjected to sensory tests, followed with gas chromatography tests to quantitively measure the aroma retention.

### Example 2: Preparation of Printed Substrate

A coarse emulsion including the ingredients listed in Table 1 was prepared. The term “wet %”, as used in this disclosure, means the weight percentage (of an ingredient) based on the total weight of the emulsion including water. Using a high shear homogenizer, the coarse emulsion was reduced to a fine emulsion. The fine emulsion was then printed on two different substrate sheets, i.e., a nonwoven polyurethane and a spunlace polyester, with a piezo-driven REA Jet valve having 350 μm nozzles.

TABLE 1

| Ingredients | Wet % |
|---|---|
| Water | 54.7 |
| OSA modified starch | 6.9 |
| Fragrance oil | 28.8 |
| Maltose | 8.6 |
| Glycerin | 1.0 |
| Sodium Lauryl Sulfate | 0.03 |
| Blue #1 | 0.005 |
| Total | 100.0 |

The printed substrate sheets were dried with infrared (IR) light for 2 minutes. The printed substrate sheets had a fragrance loading of 0.6-2.1 mg/cm² with varying dot sizes and dot-to-dot distances.

Performance of the printed substrate sheets were tested in 12-quart containers with a fragrance loading in the range of 20 mg to 80 mg. Independent of load density, all sheets released fragrance for at least 5 months with occasional opening (once per week), and for at least 3 weeks with frequent opening (opened once every day and left open for 5 minutes before being closed again), with some variation in intensity depending on substrate material, dot sizes and fragrance amount. In particular, bigger printed dots exhibited slower release at the beginning and higher retention at the end of the 5-month test.

Abrasion Tests. Following the test method of Example 1, the printed and dried fragrance emulsion showed excellent adhesion on the substrate sheets, surviving >120 strokes with 250 g of added weight.

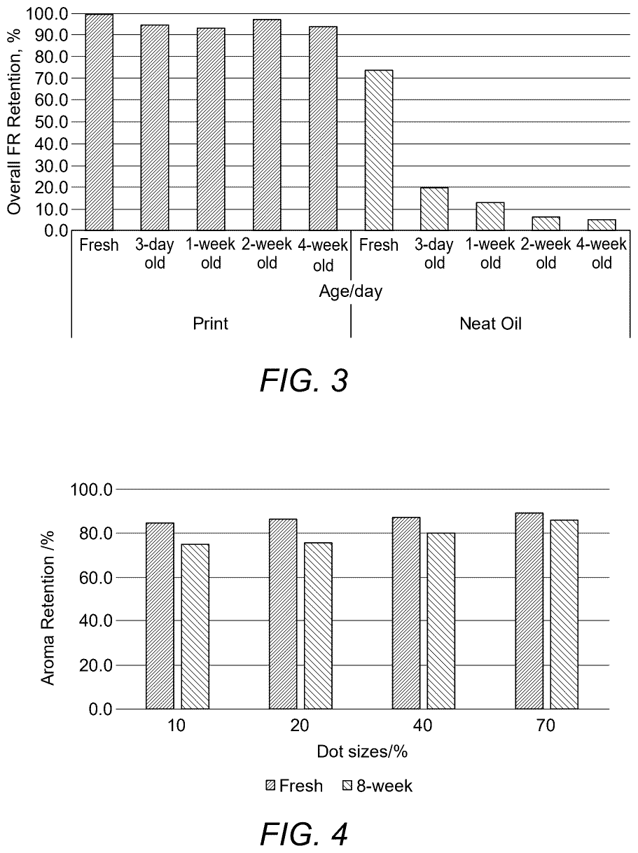

Aroma Retention Tests. Following the test method of Example 1, the printed and dried fragrance emulsion showed greatly improved overall retention of the fragrance over time compared to neat fragrance oil samples (FIG. 3). In addition, the printed and dried fragrance emulsion preserved overall fragrance very well, with a fragrance retention of >90% after 4 weeks exposure. In contrast, the neat fragrance oil samples showed a dramatic loss of overall fragrance from the first day, and the fragrance retention decreased to 20% after 3 days exposure, and 5% after 4 weeks exposure (FIG. 3).

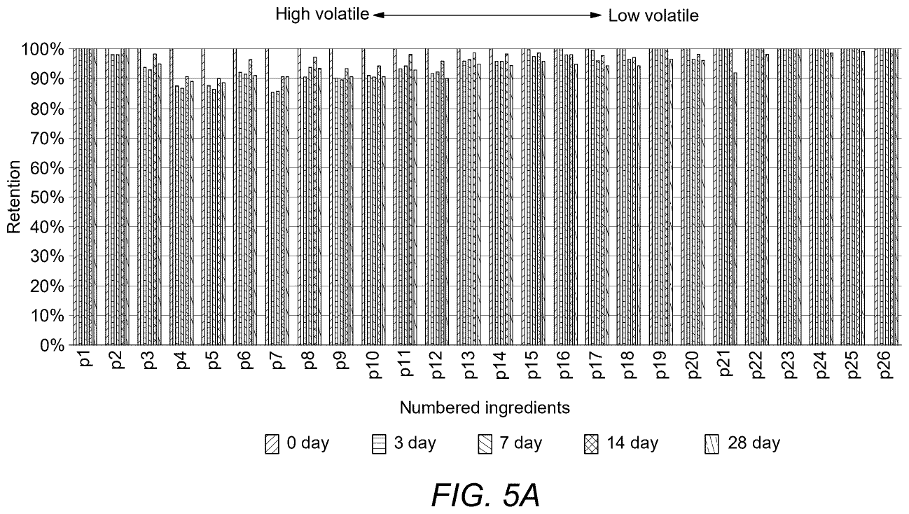

Dot size was found to affect the fragrance retention and release rate of fragrance in samples freshly prepared and after storage for 8 weeks (FIG. 4). Larger dots exhibited more protection of fragrance contained therein, and thus slowed the fragrance diffusion from the printed and dried fragrance emulsion. This was reflected in both retention and release profiles of the fragrance.

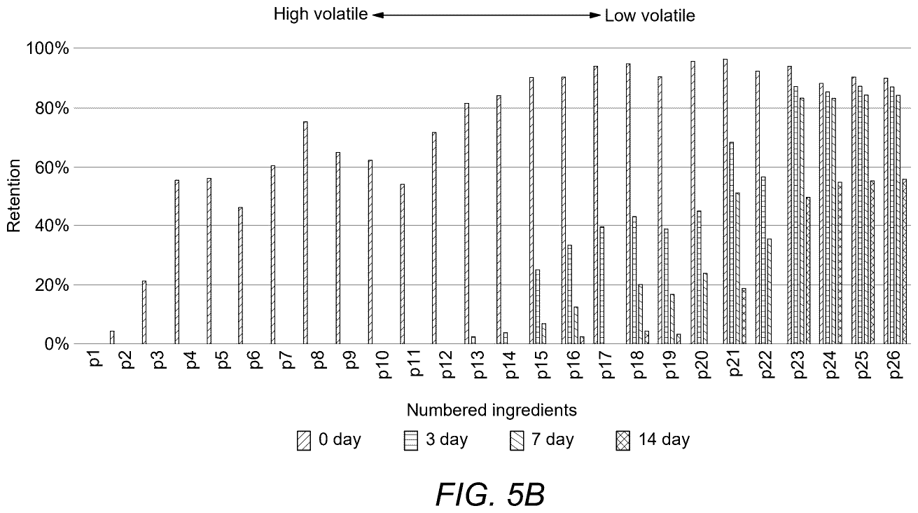

Gas chromatography (GC) analysis indicated that the printed fragrance emulsion protected highly volatile ingredients from early release (FIG. 5A) compared to neat fragrance oil sample (FIG. 5B). For neat fragrance oil sample, very volatile ingredients showed significant loss on the first day, with complete loss of the most volatile ingredient p1, only 5% retention of the very volatile ingredient p2, and 20% retention of the volatile ingredient p3. By day 3, the 12 most volatile ingredients (p1-p12) in the neat fragrance oil sample were completely lost, and the remaining ingredients (p13-p26) continued to decrease over time. After 2 weeks, only the low volatile ingredients (p23-p26) were still detected by GC in the neat fragrance oil sample, and out of 26 ingredients only 4 ingredients (p23-p26) were still retained at a retention of 50-60%. See FIG. 5B. By comparison, the printed fragrance emulsion sample showed excellent retention of all ingredients over the 4-weeks period, with only a slight loss of some of the highly volatile ingredients. See FIG. 5A.

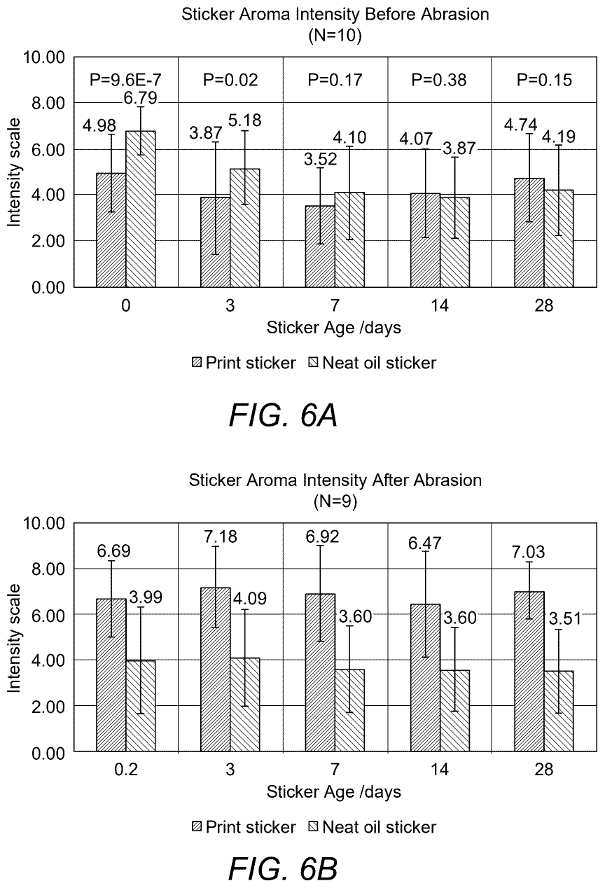

Activation Tests. Following the test method of Example 1, the printed substrate sheets were subjected to activation by abrasion. Printed fragrance emulsion samples and neat fragrance oil samples of the same age were paired for direct comparison. Before abrasion, when the samples were fresh, the neat fragrance oil samples showed much higher fragrance intensity than the printed fragrance emulsion samples, a difference that gradually decreased as exposure time increased (FIG. 6A). After 1 week, the printed fragrance emulsion samples and the neat fragrance oil samples had very close intensities, and after 2 weeks the printed fragrance emulsion samples outperformed the neat fragrance oil samples. These results demonstrated that printed fragrance emulsion provided better stability to the fragrance over time than neat fragrance oil.

Upon abrasion of the samples, it was found that abrasion activated the printed fragrance emulsion samples and released the fragrance, making the printed fragrance emulsion samples immediately smell much stronger (FIG. 6B). By comparison, the neat fragrance oil samples exhibited no change in fragrance intensity after abrasion (FIG. 6B). The abraded printed fragrance emulsion samples showed stronger fragrance intensity than the neat fragrance oil samples from the first day. Moreover, the olfactory intensities and top notes of all abraded printed fragrance emulsion samples at different exposure times were very similar. These results demonstrated the excellent retention of all volatile fragrance ingredients in the printed fragrance emulsion during the entire test period.

### Example 3: Flavor Printed on Polyester Film for Frozen Food Packaging

A flavor emulsion was prepared using the ingredients listed in Table 2. The flavor oil droplets in the emulsion had mean oil droplet size of less than 1 micron. The flavor emulsion was printed on a polyester film to form different sized dots. The printed flavor emulsion was then dried and evaluated for flavor retention. The evaluation found that larger printed dot size increased the flavor retention.

The printed flavor emulsion was also dried with air at either 67° C. or 100° C. and evaluated for flavor retention. The printed and dried flavor emulsions were evaluated at 0, 4 and 8 weeks storage time. It was found that the printed flavor emulsions, dried at either 67° C. or 100° C., maintained nearly 100% flavor retention through the 8 weeks of storage.

TABLE 2

| Ingredients | Wet % |
|---|---|
| Flavor (Sweet Orange Valencia) oil | 21.6 |
| OSA modified starch | 5.2 |
| Maltose | 7.1 |
| Water | 66.1 |
| Total | 100.00 |

### Example 4: Flavor Retention with Different Substrates

A flavor emulsion was prepared using the ingredients listed in Table 3. The flavor emulsion was printed on a PVOH (polyvinyl alcohol) film and a polyester film respectively. The printed flavor emulsions were either air-dried at room temperature or dried with infrared (IR) light. Flavor retention was assessed on the same day of printing/drying and the results (Table 4) indicated that flavor emulsion printed and dried on polyester film has significantly higher flavor retention than flavor emulsion printed and dried on PVOH film.

TABLE 3

| Ingredients | Wet % |
|---|---|
| Flavor (Sweet Orange Valencia) oil | 24.0 |
| OSA modified starch | 5.8 |
| Maltose | 7.9 |
| Water | 62.3 |
| Total | 100.00 |

TABLE 4

Flavor Retention

| Drying Method | PVOH | Polyester |
|---|---|---|
| Air | 8.2% | 83.7% |
| IR | 24.1% | 96.7% |

### Example 5: Delivery of Coffee Flavor on a Substrate

A coffee flavor emulsion was prepared using the ingredients listed in Table 5. The emulsion was printed on a polyester film and dried. A portion of the film containing 150 dots, delivering 6.7 mg of the coffee flavor oil, was taped to the lid of a coffee cup. For comparison, a control was prepared by dosing 0.67 g of 1% neat coffee flavor directly into a second cup with a blank film taped to the lid. Boiling water (100 mL) was added into each cup and the lids were secured to the cups respectively.

A trained panel of subjects evaluated the coffee aroma intensity (0-10 Scale) upon opening of the lid. The sensory evaluation results demonstrated that the printed polyester film delivered higher coffee aroma than the neat coffee flavor oil sample over the course of the evaluation (Table 6). The printed polyester film sample was perceived as significantly higher than the control at each time point.

TABLE 5

| Ingredients | Wet % |
|---|---|
| Coffee flavor oil | 21.6 |
| OSA modified starch | 5.2 |
| Maltose | 7.1 |
| Water | 66.1 |
| Total | 100.00 |

TABLE 6

| | |
|---|---|
| Time of Rating | Sensory Evaluation |

| (min.) | Control | Printed Sample |
|---|---|---|
| 0.5 | 2.8 | 4.7 |
| 2 | 3.3 | 5.2 |
| 5 | 3.3 | 4.8 |
| 10 | 2.6 | 4.9 |
| 15 | 2.9 | 4.4 |
| 20 | 3.0 | 4.3 |

### Example 6: Delivery of Chicken Flavor on a Substrate

A chicken flavor emulsion was prepared using the ingredients listed in Table 7. The emulsion was printed on a polyester film and dried. A 3″×3″ strip of the printed polyester film, delivering about 19 mg of the chicken flavor oil, was taped to the lid of a container containing 100 g of frozen cauliflower rice. For comparison, a control was prepared by dosing 0.19 g of 10% neat chicken flavor oil directly onto cauliflower rice with a blank film taped to the lid. Both the printed sample and the control sample were microwaved for 2 minutes and allowed to cool for 1 minute prior to the lid being removed (T-0) and the samples being assessed for chicken aroma intensity.

A trained panel of subjects evaluated the chicken aroma intensity (0-10 Scale) upon opening of the lid. The sensory evaluation results demonstrated that the printed polyester film delivered equal or higher chicken aroma than the control (neat chicken flavor oil sample) (Table 8).

TABLE 7

| Ingredients | Wet % |
|---|---|
| Chicken flavor oil | 21.6 |
| OSA modified starch | 5.2 |
| Maltose | 7.1 |
| Water | 66.1 |
| Total | 100.00 |

TABLE 8

| | |
|---|---|
| Time of Rating | Sensory Evaluation |

| (min.) | Control | Printed Sample |
|---|---|---|
| 0.5 | 1.9 | 2.6 |
| 2 | 1.8 | 2.8 |
| 5 | 1.8 | 3.1 |
| 10 | 2.2 | 2.9 |
| 15 | 2.4 | 2.3 |
| 20 | 2.3 | 2.3 |

### Example 7: Flavor Emulsions with Different Carriers or Different Oil Droplet Sizes

Flavor emulsions composed of 24% Sweet Orange Valencia flavor oil, 64% water, and the carriers listed in Table 9 were prepared and printed on polyester substrates. The printed flavor emulsions were dried with infrared (IR) light. Flavor retention was assessed on the same day of printing/drying and the results (Table 9) demonstrated that the flavor emulsion comprising flavor oil droplets having mean oil droplet size of less than 1 micron can significantly increase the flavor retention in the composition.

TABLE 9

| | Shear Rate | Mean Flavor Oil | Flavor |
|---|---|---|---|
| Carrier | (rpm) | Droplet Size (μm) | Retention |
| 6.5% OSA modified starch | 12000 | 2.21 | 59.3% |
| and 5.5% Maltose | | | |
| 6.5% OSA modified starch | 24000 | 0.72 | 96.8% |
| and 5.5% Maltose | | | |
| 3.2% OSA modified starch | 12000 | 2.5 | 42.7% |
| and 8.8% Maltose | | | |

The term “rpm” means revolutions per minute.

### Example 8: Flavor Emulsions Air-Dried at Different Temperatures

A flavor emulsion was prepared using the ingredients listed in Table 10. The prepared flavor emulsion comprised flavor oil droplets having a mean droplet size of 0.70 μm. The prepared flavor emulsion also had a viscosity of 10.1 cP measured at room temperature. The emulsion was printed on a polyester film and dried with air at different temperatures (Table 11). Flavor retention was assessed on the same day of printing/drying and the results (Table 11) demonstrated that high flavor retention can be achieved by air-drying the flavor emulsion at a temperature of up to 100° C.

TABLE 10

| Ingredients | Wet % |
|---|---|
| Flavor (Sweet Orange Valencia) oil | 21.6 |
| OSA modified starch | 5.2 |
| Maltose | 7.1 |
| Water | 66.1 |
| Total | 100.00 |

TABLE 11

| Air Temperature (° C.) | Flavor Retention |
|---|---|
| 67 | 94.6% |
| 83 | 102.3% |
| 100 | 96.9% |

### Example 9: Fragrance Emulsions with Different Carriers or on Different Substrates

Two fragrance emulsions were prepared using the ingredients listed in Table 12. Emulsion 1 had a viscosity of 16 cP at room temperature and Emulsion 2 had a viscosity of 17 cP at room temperature. The fragrance oil droplets present in both fragrance emulsions had the same mean oil droplet size of 0.4 micron. The emulsions were printed on polyester film and PVOH film respectively with a piezo-driven REA Jet valve having 350 μm nozzles. The printed fragrance emulsions were then dried with infrared (IR) light for 4 minutes. The retention of fragrance oil was measured fresh and after 9 days storage at 4° C. respectively. The results are shown in Table 13.

TABLE 12

Wet %

| Ingredients | Emulsion 1 | Emulsion 2 |
|---|---|---|
| Fragrance oil | 25 | 25 |
| OSA modified starch | 6.8 | 6.8 |
| Maltose | 5.7 | 8.1 |
| Water | 62.5 | 60.1 |
| Total | 100 | 100 |

TABLE 13

Fragrance Retention

| | Substrate | Fresh | after 9 days storage |
|---|---|---|---|
| Emulsion 2 | Polyester film | 92.6% | 91.2% |
| Emulsion 1 | Polyester film | 88.2% | 81.7% |
| Emulsion 2 | PVOH film | 84.3% | 78.4% |
| Emulsion 1 | PVOH film | 69.3% | 49.9% |

### Example 10: Fragrance Emulsions Printed on Different Substrates

Emulsion 2 (as in Table 12) was printed on following substrates respectively: polypropylene (PP) film, polyester film, polyurethane (PU) film, HPMC film and PVOH film. The printings were conducted with a piezo-driven REA Jet valve having 350 μm nozzles. The printed fragrance emulsions were then dried with infrared (IR) light for 4 minutes. The retention of fragrance oil was measured fresh and after 9 days storage at 4° C. respectively. The results are shown in Table 14.

TABLE 14

Fragrance Retention

| Substrate | Fresh | after 9 days storage |
|---|---|---|
| PP film | 96.6% | 96.9% |
| PU film | 91.9% | 98.4% |
| Polyester film | 92.6% | 91.2% |
| PVOH film | 84.3% | 78.4% |
| HPMC film | 85.6% | 72.5% |

### Example 11: Flavor Emulsions with Different Carriers or on Different Substrates

Two flavor (Orange Valencia) emulsions were prepared using the ingredients listed in Table 15. Emulsion 3 had a viscosity of 19 cP at room temperature and Emulsion 4 had a viscosity of 21 cP at room temperature. The flavor oil droplets present in both flavor emulsions had the same mean oil droplet size of 0.7 micron. The emulsions were printed on polyester film and PVOH film respectively with a piezo-driven REA Jet valve having 350 μm nozzles. The printed flavor emulsions were then dried with infrared (IR) light for 4 minutes. The retention of flavor oil was measured fresh. The results are shown in Table 16.

TABLE 15

Wet %

| Ingredients | Emulsion 3 | Emulsion 4 |
|---|---|---|
| Orange Valencia oil | 25 | 25 |
| OSA modified starch | 6.8 | 6.8 |
| Maltose | 5.7 | 8.1 |
| Water | 62.5 | 60.1 |
| Total | 100 | 100 |

TABLE 16

| | Substrate | Flavor Retention |
|---|---|---|
| Emulsion 4 | Polyester film | 98.6% |
| Emulsion 3 | Polyester film | 87.0% |
| Emulsion 4 | PVOH film | 70.2% |
| Emulsion 3 | PVOH film | 44.9% |

### Example 12: Flavor Emulsions Printed on Different Substrates

Emulsion 4 (as in Table 15) was printed on following substrates respectively: polypropylene (PP) film, polyester film, polyurethane (PU) film, HPMC film and PVOH film. The printings were conducted with a piezo-driven REA Jet valve having 350 μm nozzles. The printed flavor emulsions were then dried with infrared (IR) light for 4 minutes. The retention of flavor oil was measured fresh and after 13 days storage at 4° C. respectively. The results are shown in Table 17.

TABLE 17

Flavor Retention

| Substrate | Fresh | after 13 days storage |
|---|---|---|
| PP film | 98.8% | 96.0% |
| PU film | 98.1% | 103.1% |
| Polyester film | 98.6% | 92.4% |
| PVOH film | 70.2% | 44.7% |
| HPMC film | 93.4% | 70.7% |

### Example 13: Flavor Emulsions with 20% Oil and Various Maltose/Starch Ratios

Six flavor emulsions were prepared using the ingredients listed in Table 18. The term “Emul” means Emulsion. The flavor oil droplets present in all flavor emulsions had about the same mean oil droplet size of 0.4 micron. The viscosity of the emulsions was controlled in the range of from 12 to 22 cP at room temperature. The emulsions were printed respectively on polyester film with a piezo-driven REA Jet valve having 350 μm nozzles. The printed flavor emulsions were then dried with infrared (IR) light (drying time shown in Table 19). The flavor oil content in the printed and dried flavor emulsions was 20 wt % based on the weight of the flavor emulsion on a moisture-free basis. The retention of flavor oil was measured fresh and after 6-hour exposure to air in an environmental chamber set to 37° C., 70% RH. The results are shown in Table 19.

TABLE 18

| | Wet % | | | | | |
|---|---|---|---|---|---|---|
| | Emul | Emul | Emul | Emul | Emul | Emul |
| Ingredients | 5 | 6 | 7 | 8 | 9 | 10 |
| Orange Valencia oil | 5 | 5.4 | 5.4 | 8 | 8 | 10.2 |
| OSA modified starch | 20 | 20.5 | 19.4 | 16 | 9.6 | 4.1 |
| Maltose | 0 | 1.1 | 2.2 | 16 | 22.4 | 36.8 |
| Water | 75 | 73 | 73 | 60 | 60 | 48.9 |
| Total | 100 | 100 | 100 | 100 | 100 | 100 |

TABLE 19

| | | Flavor Retention | |
|---|---|---|---|
| | IR Drying Time | | After 6-hour exposure |
| | (min) | Fresh | to air at 37° C. |
| Emulsion 5 | 2 | 82.9% | 76.3% |
| Emulsion 6 | 2 | 80.5% | 86.9% |
| Emulsion 7 | 2 | 91.0% | 87.1% |
| Emulsion 8 | 2 | 86.7% | 81.4% |
| Emulsion 9 | 2 | 86.2% | 76.7% |
| Emulsion 10 | 4 | 88.6% | 76.8% |

### Example 14: Flavor Emulsions with 60% Oil and Various Maltose/Starch Ratios

Six flavor emulsions were prepared using the ingredients listed in Table 20. The term “Emul” means Emulsion. The flavor oil droplets present in all flavor emulsions had about the same mean oil droplet size of 0.4 micron. The viscosity of the emulsions was controlled in the range of from 21 to 25 cP at room temperature. The emulsions were printed respectively on polyester film with a piezo-driven REA Jet valve having 350 μm nozzles. The printed flavor emulsions were then dried with infrared (IR) light (drying time shown in Table 21). The flavor oil content in the printed and dried flavor emulsions was 60 wt % based on the weight of the flavor emulsion on a moisture-free basis. The retention of flavor oil was measured fresh and after 6-hour exposure to air in an environmental chamber set to 37° C., 70% RH. The results are shown in Table 21.

TABLE 20

| | Wet % | | | | | |
|---|---|---|---|---|---|---|
| | Emul | Emul | Emul | Emul | Emul | Emul |
| Ingredients | 11 | 12 | 13 | 14 | 15 | 16 |
| Orange Valencia oil | 23 | 23 | 24.4 | 25.2 | 27.3 | 28 |
| OSA modified starch | 15.3 | 14.6 | 14.6 | 11.8 | 9.1 | 7.5 |
| Maltose | 0 | 0.8 | 1.6 | 5.0 | 9.1 | 11.2 |
| Water | 61.7 | 61.6 | 59.4 | 58 | 54.5 | 53.3 |
| Total | 100 | 100 | 100 | 100 | 100 | 100 |

TABLE 21

| | | Flavor Retention | |
|---|---|---|---|
| | IR Drying Time | | After 6-hour exposure |
| | (min) | Fresh | to air at 37° C. |
| Emulsion 11 | 2 | 82.9% | 79.2% |
| Emulsion 12 | 2 | 82.2% | 76.6% |
| Emulsion 13 | 2 | 84.0% | 82.5% |
| Emulsion 14 | 2 | 85.0% | 83.6% |
| Emulsion 15 | 3 | 88.1% | 82.5% |
| Emulsion 16 | 3 | 86.3% | 82.3% |

### Example 15: Flavor Emulsions with 20% Oil and Various Maltose/Starch Ratios

Emulsions 5-10 (as in Table 18) were printed respectively on PVOH film with a piezo-driven REA Jet valve having 350 μm nozzles. The printed flavor emulsions were then dried with infrared (IR) light (drying time shown in Table 22). The flavor oil content in the printed and dried flavor emulsions was 20 wt % based on the weight of the flavor emulsion on a moisture-free basis. The retention of flavor oil was measured fresh and after 6-hour exposure to air in an environmental chamber set to 37° C., 70% RH. The results are shown in Table 22.

TABLE 22

| | | Flavor Retention | |
|---|---|---|---|
| | IR Drying Time | | After 6-hour exposure |
| | (min) | Fresh | to air at 37° C. |
| Emulsion 5 | 2 | 73.1% | 76.9% |
| Emulsion 6 | 2 | 85.6% | 78.3% |
| Emulsion 7 | 2 | 90.7% | 91.7% |
| Emulsion 8 | 2 | 91.4% | 91.7% |
| Emulsion 9 | 2 | 93.9% | 89.4% |
| Emulsion 10 | 4 | 92.6% | 63.4% |

### Example 16: Flavor Emulsions with 40% Oil and Various Maltose/Starch Ratios

Six flavor emulsions were prepared using the ingredients listed in Table 23. The term “Emul” means Emulsion. The flavor oil droplets present in all flavor emulsions had about the same mean oil droplet size of 0.4 micron. The viscosity of the emulsions was controlled in the range of from 12 to 24 cP at room temperature. The emulsions were printed respectively on polyester film with a piezo-driven REA Jet valve having 350 μm nozzles. The printed flavor emulsions were then dried with infrared (IR) light (drying time shown in Table 23). The flavor oil content in the printed and dried flavor emulsions was 40 wt % based on the weight of the flavor emulsion on a moisture-free basis. The retention of flavor oil was measured fresh and after 6-hour exposure to air in an environmental chamber set to 37° C., 70% RH. The results are shown in Table 24.

TABLE 23

| | Wet % | | | | | |
|---|---|---|---|---|---|---|
| | Emul | Emul | Emul | Emul | Emul | Emul |
| Ingredients | 17 | 18 | 19 | 20 | 21 | 22 |
| Orange Valencia oil | 11.4 | 12.8 | 12.8 | 16.0 | 18.0 | 20.0 |
| OSA modified starch | 17.1 | 18.2 | 17.3 | 14.4 | 10.8 | 6.0 |
| Maltose | 0.0 | 1.0 | 1.9 | 9.6 | 16.2 | 24.0 |
| Water | 71.5 | 68.0 | 68.0 | 60.0 | 55.0 | 50.0 |
| Total | 100 | 100 | 100 | 100 | 100 | 100 |

TABLE 24

| | | Flavor Retention | |
|---|---|---|---|
| | IR Drying Time | | After 6-hour exposure |
| | (min) | Fresh | to air at 37° C. |
| Emulsion 17 | 2 | 90.3% | 89.7% |
| Emulsion 18 | 2 | 91.6% | 90.7% |
| Emulsion 19 | 2 | 98.2% | 97.9% |
| Emulsion 20 | 2 | 93.7% | 94.0% |
| Emulsion 21 | 3 | 92.2% | 91.5% |
| Emulsion 22 | 3 | 92.9% | 91.4% |

### Example 17: Flavor Emulsions with 40% Oil and Various Maltose/Starch Ratios

Emulsions 17-22 (as in Table 23) were printed respectively on PVOH film with a piezo-driven REA Jet valve having 350 μm nozzles. The printed flavor emulsions were then dried with infrared (IR) light (drying time shown in Table 25). The flavor oil content in the printed and dried flavor emulsions was 40 wt % based on the weight of the flavor emulsion on a moisture-free basis. The retention of flavor oil was measured fresh and after 6-hour exposure to air in an environmental chamber set to 37° C., 70% RH. The results are shown in Table 25.

TABLE 25

| | | Flavor Retention | |
|---|---|---|---|
| | IR Drying Time | | After 6-hour exposure |
| | (min) | Fresh | to air at 37° C. |
| Emulsion 17 | 2 | 88.6% | 88.4% |
| Emulsion 18 | 2 | 88.7% | 87.5% |
| Emulsion 19 | 2 | 96.3% | 97.4% |
| Emulsion 20 | 2 | 92.0% | 92.4% |
| Emulsion 21 | 2 | 88.1% | 89.2% |
| Emulsion 22 | 2 | 89.9% | 85.1% |

### Example 18: Odor-Masking Shoe Insert

A fragrance emulsion was prepared using the ingredients listed in Table 26. The fragrance emulsion included additional additive (microcrystalline cellulose (MCC)) suspended therein. It had a viscosity of about 1200 cP at room temperature. The fragrance emulsion was printed on the surface of a spunlace polyester substrate having a silicon adhesive backing and polypropylene liner using a pneumatically driven P Jet valve having 1.1 mm nozzles, with an average wet dot weight of about 5 mg. The printed substrate was dried with infrared (IR) light for 4 minutes. The printed and dried fragrance emulsion had fragrance retention of more than 85%. The printed and dried substrate had a fragrance loading of 0.6 mg/cm² and was cut into 2-inch by 4-inch pieces to be used as shoe inserts. Each insert had fragrance oil loading of about 20 mg. The shoe insert was placed onto the midfoot area of the shoe insoles for odor masking and could last for a continuous 7-day period.

TABLE 26

| Ingredients | Wet % |
|---|---|
| Carboxymethyl cellulose | 3.45 |
| (Aqualon ™ CMC 7LXF) | |
| MCC (FLOCEL ® 101) | 2.00 |
| Sodium Lauryl Sulfate | 0.10 |
| Fragrance oil | 6.25 |
| OSA modified starch | 3.38 |
| Maltose | 2.87 |
| Maltodextrin (dextrose | 6.55 |
| equivalent (DE) 10) | |
| Glycerin | 0.4 |
| Water | 75 |
| Total | 100.00 |

### Example 19: Sleep-Aid Pillow Insert

A sleep aid fragrance emulsion was prepared using the ingredients listed in Table 27. The fragrance emulsion was printed on the surface of a sheet composed of recycled cotton with a piezo-driven REA Jet valve having 350 μm nozzles, with an average wet dot weight of about 0.45 mg. The printed fragrance emulsion was dried with infrared (IR) light for 1 minute. The printed and dried fragrance emulsion had fragrance retention of more than 90%. The printed and dried sheet had a fragrance loading of 0.7-1.2 mg/cm² and was cut into 7.4-inch by 9.7-inch pieces. Each piece had a fragrance loading of about 250-440 mg and was placed between the pillow and the pillowcase for enhanced fragrance release trigged by head movements, body temperature and/or sweating, and functioned as sleep aids.

TABLE 27

| Ingredients | Wet % |
|---|---|
| Fragrance oil | 28.8 |
| OSA modified starch | 7.26 |
| Maltose | 8.88 |
| Glycerin | 0.45 |
| Water | 54.61 |
| Total | 100.00 |

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

1. A composition comprising a substrate and an aroma emulsion printed and dried on a surface of the substrate, wherein the aroma emulsion comprises (i) an aroma, (ii) a carrier comprising a modified starch and a disaccharide and/or a disaccharide alcohol, and optionally (iii) a retention aid.

2. The composition of claim 1, wherein the carrier comprises from 5 wt % to 90 wt %, from 5 wt % to 70 wt %, from 5 wt % to 60 wt %, from 10 wt % to 70 wt %, or from 10 wt % to 60 wt % disaccharide and/or disaccharide alcohol, based on the weight of the carrier.

3. The composition of claim 1, wherein the disaccharide is selected from the group of maltose, sucrose, fructose, trehalose, lactose, and combinations thereof.

4. The composition of claim 1, wherein the disaccharide comprises maltose.

5. The composition of claim 1, wherein the disaccharide alcohol is selected from the group of isomalt, mannitol, maltitol, xylitol, lactitol, and combinations thereof.

6. The composition of claim 1, wherein the disaccharide alcohol comprises isomalt and/or maltitol.

7. The composition of claim 1, wherein the modified starch comprises an octenyl succinic anhydride (OSA) modified starch.

8. The composition of claim 1, wherein the amount of the aroma is at least 20 wt %, 25 wt %, or 30 wt %, based on the weight of the printed and dried aroma emulsion on a moisture-free basis.

9. The composition of claim 1, wherein the aroma comprises a flavor and/or a fragrance.

10. The composition of claim 1, wherein the aroma emulsion comprises the retention aid selected from the group of sugars, plasticizers, and combinations thereof.

11. The composition of claim 1, wherein the surface of the substrate is made of a polymer selected from the group of polyester, polyurethane, polypropylene, polyethylene, cellulose, and combinations thereof.

12. The composition of claim 1, wherein the aroma emulsion is an oil-in-water emulsion, the aroma is present in the aroma emulsion as oil droplets, and the oil droplets have a mean oil droplet size of less than about 1 micron.

13. The composition of claim 1, wherein the aroma emulsion is printed on the surface of the substrate in an array, wherein the array comprises a dot pattern evenly distributed and spaced center point-to-center point of from 1 mm to 10 mm between dots.

14. The composition of claim 1, wherein the substrate is single layered or multilayered.

15. The composition of claim 1, further comprising a second emulsion printed and dried on the surface of the substrate, wherein the second emulsion is printed onto or adjacent to the aroma emulsion.

16. The composition of claim 1, further comprising an adhesive cover film or backing layer on the printed surface of the substrate.

17. A method of producing the composition of claim 1, comprising:
(a) providing the aroma emulsion comprising (i) the aroma, (ii) the carrier comprising the modified starch and the disaccharide and/or the disaccharide alcohol, and optionally (iii) the retention aid;
(b) printing the aroma emulsion on the surface of the substrate; and
(c) drying the printed aroma emulsion on the surface of the substrate.

18. A consumer product or consumer package comprising the composition of claim 1.
